(19) United States
(12) Patent Application Publication
(10) Pub. No.: US 2016/0000548 A1
(43) Pub. Date: Jan. 7, 2016

(54) DEVICES, METHODS, AND SYSTEMS RELATED TO EXPANDABLE IMPLANTS

(71) Applicant: Elwha LLC, Bellevue, WA (US)

(72) Inventors: Erez Lieberman Aiden, Cambridge, MA (US); Mahalaxmi Gita Bangera, Renton, WA (US); Michael H. Baym, Cambridge, MA (US); Daniel Hawkins, Pleasanton, CA (US); Roderick A. Hyde, Redmond, WA (US); Muriel Y. Ishikawa, Livermore, CA (US); Jordin T. Kare, Seattle, WA (US); Eric C. Leuthardt, St. Louis, MO (US); Gary L. McKnight, Bothell, WA (US); Elizabeth A. Sweeney, Seattle, WA (US); Lowell L. Wood, JR., Bellevue, WA (US)

(21) Appl. No.: 14/323,397

(22) Filed: Jul. 3, 2014

(51) Int. Cl. A61F 2/00

(52) U.S. Cl. CPC A61F 2/0059; A61F 2250/001

(57) ABSTRACT

Certain embodiments described herein relate to expandable, reversible implants. In an embodiment, the implants are controllable by way of at least one biochemical, chemical, or physical means. In an embodiment, the implants are programmable and/or pre-programmed for a particular level of expansion and/or contraction. In an embodiment, the implants are controlled remotely from a control source that is external to the subject's body.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

Various embodiments described herein include devices, methods, and systems related to one or more controllable expandable implants configured for implanting into a subject's body. In an embodiment, the controllable expandable implants are reversibly expandable.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial view of a particular disclosed embodiment.

FIG. 2 is a partial view of a particular disclosed embodiment.

FIG. 3 is a partial view of a particular disclosed embodiment.

FIG. 4 is a partial view of a particular disclosed embodiment.

FIG. 5 is a partial view of a particular disclosed embodiment.

FIG. 6 is a partial view of a particular disclosed embodiment.

FIG. 7 is a partial view of a particular disclosed embodiment.

FIG. 8 is a partial view of a particular disclosed embodiment.

FIG. 9 is a partial view of a particular disclosed embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various embodiments described herein include devices, methods, and systems related to one or more controllable expandable implants configured for implanting into a subject's body. In an embodiment, the controllable expandable implants are reversibly expandable.

In an embodiment, one or more controllable expandable implants include an at least partially enclosed device containing at least one material that expands subsequent to injection of the material into the device. In an embodiment, the device is completely enclosed. In an embodiment, the device is partially enclosed. In an embodiment, the device includes an impermeable membrane. In an embodiment, the device includes a semi-permeable membrane. In an embodiment the device includes a mesh that chemically or physically combines with the material contained therein such that it is chemically or physically enmeshed or is rendered indistinguishable from the internal material. In an embodiment, the device includes a mesh that chemically or physically combines or allows for tissue ingrowth from the subject's body. In an embodiment, the internal material is configured to expand to a predetermined shape or a predetermined amount. In an embodiment, the expansion of the internal material is configured to be controlled (e.g., expanded, contracted, initiated or accelerated for either expansion or contraction) by at least one of temperature, hydration, pH, salt concentration, surface tension, specific antigen, specific chemical (e.g., protein), light, ultrasound, heat, magnetic force, microwave energy, acoustic energy, or mechanical manipulation (e.g., of the external membrane or one or more internal components).

In an embodiment, the device includes a “smart” control system configured to be informed by at least one of a preprogrammed signal or a signal detected by at least one internal or external sensor or other signal. For example, the external signal includes, but is not limited to signaling in response to one or more of a time/date signal, location (e.g., GPS) signal, specific activity (e.g., use of cell phone or computer), environmental signal (e.g., temperature, lighting, odor, sound, etc.), or physiological sensor (e.g., muscular or neuromuscular response to stimuli including, for example, muscular rigidity; biochemical/biophysical response to stimuli including, for example, blood pressure, sweat production, heart rate, etc.).

In an embodiment, the system includes control circuitry configured to control at least a portion of the device with regards to one or more movements (e.g., speed, amount, or direction of expansion or contraction) within the implantable device. In an embodiment, the control circuitry is operably coupled to at least one input device configured to input a target profile or target contour of the implantable device. In an embodiment, the input device includes at least one of a keyboard, keypad, touchscreen, voice recognition device, or other input device. In an embodiment, the control circuitry includes at least one external component that assists in the movement within the implantable device.

In an embodiment, the external component is operably coupled to at least one internal or external sensor configured to detect the amount, speed, or direction of the movement within the implantable device. In an embodiment, the external sensor includes at least one of global positioning system sensor, time or date sensor, environmental temperature sensor, light sensor, odorant sensor, auditory sensor, camera, or other environmental sensor.

In an embodiment, the system includes one or more memory devices configured to store subject information data. In an embodiment, the stored subject information data includes at least one of data corresponding to at least one of a past state of the implantable device, a pre-programmed profile of the implant, a current state of the implantable device, or a customized profile of the implant provided by the subject or the subject's healthcare worker. In an embodiment, the control circuitry is configured to write to the implantable storage device with subject information data, including one or more of an updated status of the implantable device during or subsequent to expansion or contraction.

In an embodiment, the device includes a flexible body. In an embodiment, the device includes one or more rigid portions. In an embodiment, the device is elongate, cuboidal, spherical, elliptical, pyramidal, or planar. In an embodiment, the device is customized for at least one of size or shape, depending on the use and/or location in the subject's body. In an embodiment the device has a first configuration and at least one second configuration. In an embodiment, the second configuration can be a maximum size or shape allowable by constraints of the device or can be at any point from the first size or configuration to the maximum size or shape. A second configuration can differ in size or shape upon subsequent uses.

In an embodiment, the device includes a cavity and a port for transferring one or more materials into the device before, during, or after implanting into a subject's body. In an embodiment, the port includes a valve. In an embodiment, the device is configured to be injected with one or more materials. In an embodiment, the device is transformable from a first configuration to a second configuration by introduction of one or more materials into the flexible body, e.g., from a reservoir or a cartridge. In an embodiment, the valve is accessible by way of small incision in the subject's body to the location where the implant resides, thus overcoming the need for invasive surgery to access the implant once it has been placed in the subject's body. See, for example, U.S. Pat. No. 4,969,899, which is incorporated herein by reference.

In an embodiment, the device includes two or more sections (e.g., layers, compartments such as a honeycomb structure, segments, sections, etc.) that may be wholly separable and independently controllable (e.g., separately fillable or manipulatable). In an embodiment, the layers may include concentric layers separated by a larger amount of material (e.g., stacked) or layers separated by a small amount of filler material (e.g. sleeves within sleeves). In an embodiment, the two or more sections are separated by at least one barrier. In an embodiment the at least one barrier is pierceable and may be manipulatable before, during, or after implanting into a subject's body. In an embodiment, the device is manipulatable in order to achieve a desired contour, profile, or firmness. In an embodiment, the device is configured in a sheet arrangement with separate sections that are independently manipulatable and/or fillable. In an embodiment, the device is configured with separate sections overlapping and movable with relation to each other. For example, the sections are configured in a petal arrangement, able to expand or collapse e.g., in a radial fashion. For example, the sections are configured in a telescoping arrangement, able to expand or collapse e.g., in a longitudinal fashion. In an embodiment, the device is configured as a linear or circular segmented arrangement with separate sections that are independently manipulatable and/or fillable.

In an embodiment, each section can be fully sealed from each other, or include one or more of a suture, clip, elastomeric band, or biasing element. In an embodiment, the device includes two or more separate sections. In an embodiment, at least one section remains unfilled/unmanipulated or only partially filled or manipulated and may be filled/manipulated for expansion at a later date. In an embodiment, the device includes about 1, 2, 3, 4, 5, 6, 7, 8, 9, 10, 12, 14, 16, 18, 20, 25, or more separate sections. In an embodiment, the section are any size or shape (e.g., square, circular, ovoid, rectangular, elongate, triangular, amorphous, pyramidal, etc.).

In an embodiment, the device is at least partially coated with one or more substances prior to implanting into a subject's body. In an embodiment, the coating includes any biocompatible substance. In an embodiment, the coating includes a biodegradable substance. In an embodiment, the coating includes a biologically active substance. For example, in an embodiment, the coating includes at least one growth factor (e.g., fibroblast growth factor), anti-inflammatory agent (e.g., corticosteroid), antibiotic, anesthetic (e.g., lidocaine, procaine, marcaine, etc.), angiogenic inhibitor (e.g., TNP-470), tissue adhesive (Dermabond™, Focalseal™, fibrin glue, etc.) or heparin.

In an embodiment, the device includes at least one frame made of one or more of a metallic frame, Nitinol™ frame (optionally with polymer coating), metal (e.g., silver, gold, platinum, stainless steel, nickel, titanium, or an alloy thereof), e-PTFE, PTFE, polypropylene, polyacrylamide, polyurethane, silicone, polymethylmethacrolate, Dacron™, or a combination of nylon and dorlastan fabric (e.g., 92% nylon, 8% dorlastan).

In an embodiment, the device includes an outer shell of one or more biocompatible materials. In an embodiment, the device includes an outer shell of one or more non-biodegradable materials. In an embodiment, the device includes an outer shell of one or more thermoplastic polymers. In an embodiment, the device includes an outer shell of one or more of polypropylene, silicone elastomer, PTFE, e-PTFE, polyacrylamide, polyurethane, silicone, polymethylmethacrolate, or Dacron.

In an embodiment, fibrous tissue ingrowth is desired, and the at least a portion of the outer shell of the device includes a pore size of about 1 micron to about 100 microns or any value therebetween.

In an embodiment, the device includes at least one rigid material. For example, the device includes one or more rigid material forming an adjustable member, which may comprise a single portion or multiple sections in overlapping and movable with relation to each other. For example, the sections can be configured in a petal or telescoping arrangement, able to expand or collapse e.g., in a radial, longitudinal, or other pattern. For example, the rigid material in a single or multiple sections may be driven by a motor. In an embodiment, the rigid material can be a metal or a plastic. The metal may be silver, gold, platinum, stainless steel, nickel, titanium (e.g., Nitinol™), or any alloy, and may be coated with a biocompatible coating.

In an embodiment, the device includes at least one filler material. In an embodiment the filler material includes a reversible material, such as a thermoreversible gel or hydrogel, for example, that is in a semi-solid phase at body temperature but upon cooling to a temperature below threshold level, the gel is liquefiable for reshaping of the contour (e.g., PEG or poly(vinyl alcohol), NiPAAm [poly(N-isopropyl acrylamide)], degassed distilled water). See for example, U.S. Pat. No. 7,160,931; U.S. Pat. No. 7,708,979; and U.S. Pat. App. Pub. No. 2007/0110784, each of which is incorporated herein by reference. Another example of a thermal responsive hydrogel includes a reverse thermally viscosifying polymer, for example a linear block copolymer (e.g., poloxamer). For example, another particular thermal responsive hydrogel includes poly(vinylcarboxylic acid) and a polyoxylalkylene (e.g., a triblock polymer of polyoxyethylene and polyoxypropylene). See for example, U.S. Pat. No. 7,008,628, which is incorporated herein by reference. As another example, a foam filler material can be controlled by pH within the device for expansion or contraction. See for example the topical foam of U.S. Pat. App. Pub. No. 2008/0206159, which is incorporated herein by reference. In an embodiment, a polymeric hydrogel such as methyl methacrylate combined with N-vinylpyrrolidone can be utilized with various embodiments disclosed herein. See, for example, U.S. Pat. App. Pub. No. 2009/0099655, which is incorporated herein by reference.

In an embodiment, the device includes at least one energy-responsive metallic substance. In an embodiment, the device includes a shape-memory alloy that maintains a first shape in the absence of an energy stimulus, but can be altered to a new shape upon application of an energy stimulus, e.g., when heated by an electric field. In an embodiment, the device includes at least one electroactive polymer. In an embodiment, a responsive filler material undergoes a phase change (e.g., from liquid to gas or a fluid with different vapor pressure) for varying expansion or contraction, as described herein.

In an embodiment, the filler material includes a material with latchable control of densification or stiffness upon application of transdermal energy following implantation of the device into the subject's body. For example, application of light, (infrared, visible, ultraviolet), microwave energy, acoustic energy (e.g., radio waves, ultrasound), thermal energy, magnetic energy or other energy can alter the material. For example, the reaction can alter at least one of density or stiffness by way of, for example, polymerization, gelation, chemical reaction, etc. For example, thermal energy can heat the material, causing a temperature-based, reversible reaction. For example, optical light drives a photo reaction that alters the density or stiffness via gelation, polymerization, chemical reaction, etc.

In an embodiment, the device includes at least one microencapsulated reactive compound (optionally co-mixed with injected filler material) that reacts with at least one other component to change the density or stiffness of the material. The microencapsulated compound may, for example and without limitation, be a biological or chemical compound selected from the group consisting of water, saline, an acid, a base, an enzyme, a protein, a modified protein, a peptide, a modified peptide, an oligonucleotide, a nucleotide and an aptamer. In some embodiments, microcapsules are ruptured in response to exposure to electromagnetic radiation, exposure to heat, or exposure to acoustic energy, thereby resulting in the release of the chemical or biological compound. For example, microcapsules may hold an acidic compound that, when released upon exposure to a stimulus (e.g., ultrasound), acts on a pH-responsive compound. To reverse the process, a second, more basic compound may be encapsulated in a shell that releases its contents when stimulated by a second stimulus (e.g., a different frequency of ultrasound or an RF signal). The device may include a number of microcapsules or nanocapsules so that only a portion of the capsules are triggered upon any activation. The encapsulated compound may be resident directly in a filler material or may be held in a reservoir, which may be refillable by injection.

In an embodiment, the filler material includes at least one “smart” polymer including, for example, one or more of antibodies, protein A, streptavidin, or enzymes. For example, bioconjugates can be prepared and adapted by random polymer conjugation to a specific amino group (e.g., lysine) on the protein surface or by site-specific conjugation of the polymer to specific amino acid sites (e.g., cysteine sulfhydryl groups) that have been engineered into the protein. See for example, Hoffman, Clin. Chem., 46:9 (2000) pp. 1478-1486, which is incorporated herein by reference. In an embodiment, the resulting “smart” polymer is controllable by one or more of energy, temperature, pH, or light.

In an embodiment, the filler material includes one or more nanocomposites that are chemoresponsive or include morphing mechanical behavior. For example, cellulose nanofibers or aqueous dispersions of poly(acrylic acid)-coated carbon nanotubes with properties of variation in viscosity with varying pH. See for example, Capadona et al., Science, 319:1370-1374 (2008), which is incorporated herein by reference.

In an embodiment, the filler material includes at least one photoresponsive polymer with a photoregulatable enzyme switch. For example, a “smart” polymer chain coil can be adapted that regulates substrate access and enzyme activity when it is conjugated to the enzyme at a specific position outside of the active site. See for example, Shimoboji et al., PNAS Vol. 99, 26 (2002) pp. 16592-16596, which is incorporated herein by reference. In this way, the photoresponsive polymers serve as both antennae and actuators that reversibly respond to distinct optical signals that switch the polymer-enzyme conjugates to active or inactive states, and are functional when the conjugate is either free in solution or immobilized on a substrate, such as a magnetic bead. Id.

In an embodiment, the filler material includes at least one stimuli-responsive hydrogel that responds to at least one of pH, temperature, solvent composition, electric fields, magnetic fields, or chemical or biological agent, (such as saccharides, or antigens). For example, a hydrogel prepared by grafting an antigen and corresponding antibody to a polymer network, such that the binding between the two introduces crosslinks in the network and a change in volume upon competitive binding of the free antigen breaking the non-covalent crosslinks. In an embodiment, the filler material includes a hydrogel that undergoes a state change by infiltration or exfiltration of salt compounds, from or into a reservoir of the device or from or into the surrounding tissue. In an embodiment, a pump such as an osmotic pump can motivate saline fluids to and from a reservoir or local tissues out of or into the device. In an embodiment, the filler material includes at least one “smart” hydrogel engineered to undergo a conformational change in response to a stimulus. For example, the conformational change can manifest in at least one of actuation, catalytic or signaling event, movement, swelling, interaction with other proteins, etc. which is initiated by a recognition event that translates into a mechanical action. Examples of stimuli-responsive, smart gels can be found in Miyata et al., Nature, 399, pp. 766-769 (1999); Hoffman, Clin. Chem., 46:9 (2000) pp. 1478-1486; and Ehrick et al., Nature Mat. 4 (2005) pp. 298-302, which are incorporated herein by reference. For example, a hybrid material containing genetically engineered proteins within hydrogels that produce a mechanical effect as a result of an induced conformational change and binding affinity of the protein to the stimulus can be adapted for use with various embodiments disclosed. In an embodiment, a particular “smart” hydrogel exhibits one, two, three, or more specific swelling stages in response to various ligands for fine tuning of the swelling effect. Thus, several different stimuli are utilized for engaging the “smart” polymer at various stages of swelling. Id.

In an embodiment, the filler material includes at least one “smart” porous hydrogel thin film is utilized. For example, a hydrogel structured for swelling or contracting of the films results in a tunable closing or opening of the film's pores. See for example, Tokarev et al., Adv. Mater. (2010), XX, pp. 1-17, which is incorporated herein by reference.

In an embodiment, the amount, type, and/or spatial location of the transdermal energy application is controlled by way of feedback (e.g., based on the present property or contour of the implanted device versus a desired property or contour) and can be a one-step process or an iterative process, as described herein.

In an embodiment, the filler material is responsive to some form of energy and remains in its unaltered state within the implantable device until application of energy. The form of energy may be, for example, light energy, thermal energy, electrical energy, electrochemical energy, magnetic energy, electromagnetic energy, or acoustic energy. Examples of stimuli-responsive materials and their responsive properties are provided in Bawa et al., Biomed. Mater. 4, 022001, (15 pp), (2009), which is incorporated herein by reference. The energy may be provided by an energy source, for example a light source, thermal source, electric source, electrochemical source, magnetic source, electromagnetic source, or acoustic source. The energy source may be internal to the subject as part of the device. The energy source may be external to the subject and the energy provided transdermally.

In an embodiment, the filler material changes to a different state by application of energy to drive the material to a different stiffness or density. For example, the filler material can include a thixotropic material that is softened by application of ultrasound, thereby allowing for alteration of the material. The thixotropic material then returns to its more stiffened state when the ultrasound is removed. For example the thixotropic material can be an electrorheological fluid that changes viscosity application of an electric field and returns to its original state in the absence of an electric field. While in a softened state the material may be mechanically manipulated within the device, for example between portions of the device thereby changing the shape of the device.

In an embodiment, the material includes a magnetically responsive material (e.g., ferromagnetic or paramagnetic) that is able to alter latchable states by application of magnetic fields. For example, the magnetically responsive material is altered by application of the correct magnetic field. The magnetically responsive material may include, for example, a ferrofluid, magnetorheological fluid, magnetic polymer, magnetic inorganic material, magnetically modified biological structure, magnetic particles with bound biomolecules, nanocomposite, polymer, gel, or elastomer. See, e.g., Safarik, Solid State Phenomena Vol. 151, pp 88-94, (2009) and Filipcsei et al., Advances in Polymer Science, 206:137-189, 2007, which are incorporated herein by reference. In an embodiment, a magnetically responsive material may be altered by exposure to a magnetic or electromagnetic field so as to move within the device. For example, a ferrofluid or magnetorheological fluid or nanocomposite material could be motivated to move from a reservoir to an adjustable member for expansion, then be moved back into the reservoir for contraction. For example, a magnetically responsive material, e.g., a magnetically responsive polymer or gel, having swelling properties may expand when exposed to a magnetic field. See, e.g., Filipcsei et al., idem.

In an embodiment, the filler material is segregated from the embedding tissue by at least one of a flexible enclosure, surface tension, or other physical or chemical barrier.

In an embodiment, a hydrogel including a cyclodextrin and an amphiphilic copolymer with an A polymer block (e.g., poly(alkylene oxide)) and B polymer block (e.g., poly(hydroxyalkanoate)) can be utilized with one or more embodiments disclosed herein. See for example, U.S. Pat. No. 7,297,348, which is incorporated herein by reference. In an embodiment, a thermo-reversible gel (e.g., methylcellulose) with at least one thixotropic property-increasing substance such as sugar alcohol, lactose, carmellose, or cyclodextrin that alters the viscosity of the gel while stressed (e.g., temperature range) and then alters under non-stress conditions. See for example, U.S. Pat. App. Pub. No. 2006/0211599, which is incorporated herein by reference.

In an embodiment, a covalently crosslinked hydrophilic polymer (e.g., poly(vinyl pyrrolidone) and methacrylate) or synthetic polymers (e.g., poly(N-alkylacrylamides) can be adapted for use in various embodiments disclosed herein. See for example, U.S. Pat. App. Pub. No. 2008/0132936, which is incorporated herein by reference. For example, certain pH-responsive polymers have a low viscosity at acidic or basic pH and exhibit an increase in viscosity upon reaching neutral pH, for example, due to decreased solubility. See for example, Id. For example, in an embodiment, the thermoreversible gel includes one or more of polyethylene glycol, poly(oxyethylene)-poly(oxypropylene) or an acrylate. See for example, Id.

In an embodiment, a filler material includes a gadolininium base, methylxanthine, and an N-acetylcysteine. See for example, U.S. Pat. App. Pub. No. 2009/0157069, which is incorporated herein by reference.

In an embodiment, a thermo-responsive polymer is utilized as described herein. For example, a polymer or copolymer, either synthetic or natural, that is not plastically expandable at normal body temperature but is thermo-mechanically expandable at an elevated temperature above normal body temperature can be utilized. See for example, U.S. Pat. App. Pub. No. 2011/0022148, which is incorporated herein by reference. For example, a filler material can be composed of one or more of the following materials including, polyhydroxyalkanoates, polyalphahydroxy acids, polysaccharides, proteins, hydrogels, lignin, shellac, natural rubber, polyanhydrides, polyamide esters, polyvinyl esters, polyvinyl alcohols, polyalkylene esters, polyethylene oxide, polyvinylpyrrolidone, polyethylene maleic anhydride and poly(glycerol-sibacate), optionally including poly-L-lactide, poly-.epsilon.-caprolactone or a biological fluid in the solid state such as blood plasma. See Id. In an embodiment, an implant having a plurality of particles dispersed therein is configured to have a first material property when implanted in tissue at normal body temperature and variable material property at an elevated temperature above normal body temperature. See Id. In this way, exposing the implant to electromagnetic radiation results in the incident radiation converted into heat energy thus raising the temperature of the implant above normal body temperature and thereby changing the material property relative to the first material property. See Id.

In an embodiment, the implantable device includes one or more microcapsules or microspheres that allow for expansion of the device upon release of the contents of the microspheres, for example when exposed to a transdermal energy source. In an embodiment, the rate of expansion of the device is directly proportional to the number of microcapsules or microspheres that are ruptured.

In an embodiment, the filler material includes at least one reversibly expandable sponge or spongy material, for example, that is hydrated (for example by means of a pump) once it is implanted into the subject's body. See for example, U.S. Pat. App. No. 2002/0091443, which is incorporated herein by reference.

In an embodiment, the filler material includes at least one reversible gelling polyurethane polymer with a polyalkylene oxide backbone with one or more copolymers having on average about 65 to about 95 mole % ethylene oxide monomers and at least about 5 to about 35 mole % propylene oxide monomers, and less than about 5% of any other monomer, having an average functionality of greater than 2 active isocyanate groups per prepolymer molecule in a polyurethane solution that gels when it is not sheared, and becomes fluid under shear. See for example, U.S. Patent App. Pub. No. 2009/0012462, which is incorporated herein by reference.

In an embodiment, the filler material includes a crosslinked hydrogel that can be reversibly hydrated (for example by means of an pump) following implantation into the subject's body. See for example, U.S. Pat. App. Pub. No. 2001/0046518, which is incorporated herein by reference.

In an embodiment, the implantable device is configured to change to a commanded state quickly, then subsequently revert to resting state over a longer period. For example, the initiation signal may signal a first stimulus to act quickly and when signaling for the reversal state use a more gradual signal over a longer period of time or in a more passive manner.

In an embodiment, the device is an injectable device. In an embodiment, the device is dimensioned to fit through a channel having an access gauge in the range of about 12 gauge to about 22 gauge. In an embodiment, the channel includes at least one of a needle, cannula, or catheter.

In an embodiment, the device has a wall thickness of about 1 nanometer to about 1 micrometer, or any value therebetween. In an embodiment, the device has a wall thickness of about 1 micrometer to about 1 millimeter, to about 1 centimeter, or any value therebetween.

In an embodiment, the device has an inflated diameter or length of about 1 nanometer to about 1 micrometer to about 1 millimeter to about 10 centimeters, or any value therebetween.

In an embodiment, the device has a volume of about 10 cc to about 600 cc, or any value therebetween.

In an embodiment, the device is implanted into a subject's body. In an embodiment, the device is placed in the subject's body in one or more area, including the face (e.g., chin, cheek, jaw, lips, facial fold, forehead, nose), breast (e.g., subglandular or subpectoral), chest (e.g., subpectoral), buttocks (e.g., gluteal), legs (e.g., calf, thigh), arms (e.g., bicep, tricep), genital area (e.g., construction or reconstruction of genitalia), hands, feet, stomach, or heart. In an embodiment, the device is implanted into a subject's body for construction or reconstruction of one or more body parts (e.g., as a result of amputation, injury or burn, scarring, congenital malformation, or disease). In an embodiment, the device is implanted into a subject's body for temporary alteration of one or more body parts (e.g., to temporarily alter facial appearance).

In an embodiment, the device is implanted into a subject's body subdermally. In an embodiment, the device is implanted into a subject's body submuscularly. In an embodiment, the device is implanted into a subject's body subcutaneously. In an embodiment, the device is implanted into a subject's body beneath one or more fat layers.

In an embodiment, the device is implanted into a subject's body for cosmetic purposes. In an embodiment, the device is implanted into a subject's body for comfort purposes. In an embodiment, the device is implanted into a subject's body for health treatment purposes (e.g., heart, stomach, etc.).

In an embodiment, the device is placed in the stomach of a subject in order to reduce the volume of the stomach to assist in weight control of the subject. In an embodiment, the device is placed in the heart of a subject in order to assist in decreasing volume of a chamber of the heart (e.g., in cases of heart failure) or in increasing pressure on one or more walls of the heart (e.g., in cases of heart failure).

In an embodiment, the device is placed beneath the skin to treat skin contour deficiencies due to aging, environmental exposure, weight loss, surgery, child bearing, disease, congenital malformation, or for enhanced beauty (e.g., for treating frown lines, worry lines, wrinkles, crow's feet, facial scars, acne marks, marionette lines, or for augmentation of various facial features).

In an embodiment, the subject includes a mammal, bird, fish, amphibian, or reptile. In an embodiment, the subject includes an animal used in exhibition. In an embodiment, the subject includes a human.

In an embodiment, various methods are provided for enhancing tissue in a subject's body. In an embodiment, the tissue is enhanced in at least one of firmness, contour, profile, or comfort for the subject. In an embodiment, a needle is inserted into the tissue, optionally while passing a guidewire (e.g., suture, metal filament, etc.) through the needle, which allows for the removal of the needle while a catheter is passed over the wire. The device is inserted through the catheter into the subject's body, and the catheter is withdrawn over the device, with the device remaining in the subject's body with the removal of the catheter.

In an embodiment, the device is injected directly into the subject's body with a needle inserted into a tissue of the subject's body and the forward pressure on the system maintains the device within the subject's body while the needle is withdrawn. In an embodiment, the device is surgically implanted into the subject's body by incision and suture at the desired location. In an embodiment, a port of the device is able to be accessed for injection of filler material into the device following injection or implantation into the subject's body.

In an embodiment, one or more ports are affixed to one or more ends of the device, or along any portion of a wall of the device (e.g., multiple layers include multiple walls upon which a port may be affixed). In addition, one or more ports may be included at a plurality of locations within the device where sections of the device include one or more walls. In an embodiment, the one or more ports include at least one valve (e.g., for the reservoir or cartridge and adjustable member, see Figures). In an embodiment, the valve includes at least one micro-electro-mechanical valve. In an embodiment, the valve includes an osmotic valve (e.g., allows for intake of fluid). As described elsewhere herein, control circuitry is operably coupled to the valve of the reservoir or cartridge. Also as described herein elsewhere, each port of the device can include control circuitry operably coupled to the port for precise control.

In an embodiment, the filler material includes one or more substances that may be in various physical states or combinations thereof, including, for example, non-viscous liquid, viscous liquid, gel, powder, beads, flakes, foam, continuous our non-continuous fibers, coils, fiber balls, knit fibers, woven fabric, filaments, or the like.

In an embodiment, the filler material includes one or more carrier including, for example, polyvinylpyrrolidone, silicone oil, vegetable oil, saline, gelatin, collagen, autologous fat, hyaluronic acid, autologous plasma, water, saline, silicone, carbon dioxide, or other physiological carriers.

In an embodiment, one or more gas cartridges are included in the device. In an embodiment, the one or more gas cartridges may include a time release or expiration time that allows for a certain time period of activation, and then reversal. For example, a carbon dioxide cartridge may be activated by energy released from a remote control. For example a radiofrequency device may signal the cartridge to release carbon dioxide at a specific quantity (e.g., 5 cc quantities over a 12 hour period, etc.). In an embodiment, the remote control is powered by at least one of acoustic energy, radiofrequency energy, thermal energy, or light energy.

In an embodiment, the device includes one or more motors for control of expansion or contraction of the device. In an embodiment, the one or more motors are operably connected to control circuitry, which may be programmable for operating the one or more motors. In an embodiment, the control circuitry or the one or more motors are configured to be signaled remotely.

In an embodiment, the filler material is capable of altering its phase state for manipulation of the device, including for example, filling an implantable device with a liquid that is stimulated to form a gel, and then stimulated to reversibly form a liquid for further manipulation of the device once it is implanted.

In an embodiment, the filler material includes one or more microcapsules that are responsive to a catalyst (e.g., ultrasound, etc.) and expansion of the device is controlled by the number of microcapsules that are ruptured or inflated in the device by the catalyst.

In an embodiment, the device is powered by an isobarically pressured sac with internal linear motors or attached cables or other source.

In an embodiment, a method for augmenting or shaping tissue in a subject's body includes identifying a location of the subject's body desired to be augmented or shaped, and introducing the device into said location. In an embodiment, measurements of the location and/or desired bulk of the device after manipulation are conducted. The device may be manipulated before, during, or after placing into the subject's body, as described herein. In an embodiment, the device is manipulated both before and after being placed into the subject's body. In an embodiment, the device is manipulated only one of before or after being placed into the subject's body. In an embodiment, the device is manipulated during the procedure of placing the device into the subject's body. In an embodiment, manipulating the device includes at least one of inserting filler material (e.g., solid, liquid, gas, polymer or other material that changes state, etc.) into at least one cavity of the device, or engaging one or more electrical and/or mechanical components in order to alter the configuration of the device.

In an embodiment, the device is able to be manipulated by at least one of varying volume, or expanding one or more mechanical components. In an embodiment, a filler material is able to be activated using external energy (e.g., laser, ultraviolet, microwave, magnetic field, etc.). In an embodiment, the device includes laser-driven expanding spheres. In an embodiment, the device can utilize the flow of fluid (e.g., a gas or liquid) for expansion or contraction of the device. In an embodiment, the device is configured for in vivo reshaping or in vivo resizing. In an embodiment, external energy is utilized to induce a change in volume, shape, color, or tone of the device in vivo.

As described, at least one filler material for the device includes a polymer or other material that changes state (e.g., from liquid to solid or from liquid to gel matrix, etc.) upon activation by at least one of temperature, hydration, pH, salt concentration, surface tension, specific antigen, specific chemical (e.g., protein), light, ultrasound, heat, magnetic force, or mechanical manipulation (e.g., of the external membrane or one or more internal components), as described herein.

In an embodiment, the device is configured to be manipulated over time and/or continuously following being placed into a subject's body. For example, in an embodiment, a subject or healthcare worker can adjust the contour of the device by way of manipulating at least one of volume, electrical and/or mechanical components of the device subsequent to being placed into the subject's body. For example, if the subject desires that the implant become firmer for a particular time period, the subject adjusts the implant (e.g., by remote control) to the desired shape or firmness and then when the time period has passed adjusts the implant again to the original state.

In an embodiment, the device includes an external control (e.g., remote control) that can optionally receive and process signals from one or more internal or external sensors that are operably coupled to the external control. In an embodiment, the sensor includes a spatial sensor. In an embodiment, the sensor includes a temporal sensor. In an embodiment, the sensor includes an external and/or remote sensor for detecting environmental conditions. In an embodiment, the sensor includes an internal sensor for detecting parameters or conditions of the device itself, or parameters or conditions of the subject's body in which the implantable device is implanted.

In an embodiment, the device includes a receiver and internal control circuitry. In an embodiment, the device includes a receiver and internal control circuitry that can receive signals from an external source, such as a remote control or computer or external sensors. In an embodiment, the device includes a receiver and internal control circuitry that can receive signals from one or more internal sensors that are operably coupled to the receiver and internal control circuitry. In an embodiment, the internal control circuitry is operably connected to other parts of the device. The internal control circuitry processes received signals and informs the other parts of the device, for example motors, canisters, reservoirs, valves, etc.

In an embodiment, the sensor includes an internal sensor for detecting parameters or conditions of the device itself, or parameters or conditions of the subject's body in which the implantable device is implanted. Examples of internal sensors that can be adapted for use with various embodiments described herein can be found, for example, in U.S. Patent App. Pub. No. 2012/0157804, which is incorporated herein by reference.

In an embodiment, the sensor includes an external sensor for detecting parameters or conditions external to the device and/or to the subject's body. For example the external sensor may be used to detect temperature, date, time, location, etc. as described herein.

In an embodiment, the device includes a plurality of concentric rings that form the walls of various sections of the device and that are structurally aligned to collapse or telescope as the device expands or retracts.

In an embodiment, the system includes a device with circuitry that is programmable and/or can be pre-programmed prior to implantation or subsequent to implantation, such that one or more tissue profiles can be programmed for the implant to contour itself to the profile or the implant can be customized as the subject desires. In an embodiment, the device includes one or more sections as described here, and the one or more sections can be programmed or pre-programmed separately or sequentially to attain a desired overall profile or sectional contour.

In an embodiment, one or more pre-programmed or programmable tissue profiles can be included in the system, for example, an increase in expansion at a particular time of day or on a particular day or other timed schedule. Likewise, in an embodiment, for example, the profile includes an increase in contraction at a particular time of day or on a particular day or other timed schedule. In an embodiment, the profile includes one or more options for changing at least one of the size or shape of the implantable device. In an embodiment, the profile includes one or more options for increasing the firmness of the implantable device. In an embodiment, the profile includes one or more options for altering the color of the implantable device (e.g., by altering pH). In an embodiment, the profile includes at least one option for varying different sections of the implantable device or for establishing or maintaining a bistable configuration.

In an embodiment, the implantable device includes a controllable, reversibly expandable device including a reservoir and a pump (e.g., an osmotic pump and/or osmotic valve) that provides a fluid ingress from the reservoir or from the subject's own body to an adjustable member of the device. In an embodiment, the device adjustable member includes at least one filler material as described herein that is structurally configured to swell by osmotic gradient.

In an embodiment, the implantable device includes a power source. For example, the implantable device may include at least one battery, fuel cell, wireless power transmission coil, or energy harvester. In an embodiment, the power source may include one or more microbattery or thin-film battery; see, e.g., U.S. Pat. No. 5,338,625, Thin film battery and method for making same, which is incorporated herein by reference. In an embodiment, the implantable device may include one or more power source that is rechargeable by an external source; see, e.g., U.S. Patent App. Pub. No. 2005/0143787, Method and system for providing electrical pulses for neuromodulation of vagus nerve(s), using rechargeable implanted pulse generator, which is incorporated herein by reference. In an embodiment, the power source includes a wireless transmission coil with an inductive component. In an embodiment, the power source includes an energy harvester able to harvest energy from body heat or motion. The power or energy harvester may be include a thermoelectric component or a piezoelectric component. See, e.g., U.S. Patent App. Pub. No. 2013/0041235, and U.S. Patent App. Pub. No. 2012/0157804, each of which are incorporated herein by reference.

In an embodiment, the implantable device is powered by an external power source. For example, an external power source may include at least one of an battery, a fuel cell, a wireless inductive transmission coil or an energy harvester. In an embodiment, the external power source is housed in a garment or wearable item (e.g., clothing such as a bra, bracelet, etc.).

In an embodiment the implantable device includes an energy source for providing an energy stimulus. The energy source can be, for example, a light source, thermal source, electric source, electrochemical source, magnetic source, electromagnetic source, or acoustic source. For example the energy source provides energy in the form of light (e.g., visible light, infrared light, or ultraviolet light), heat, electrical energy, magnetic energy, or acoustic energy (e.g., sound waves, ultrasound waves, microwaves, radio waves).

In an embodiment, the energy source is internal to the subject. For example the energy source may be resident to the device. For example, the internal energy source can include one or more electrical, electrochemical, or electromagnetic source comprising microcircuitry. For example the internal energy source can include a heater. Exemplary heaters include passive heaters, resistive heaters, and active heaters. For example a resistive heater or a passive heater may be responsive to absorption of electromagnetic radiation from microcircuitry or from an external energy source. In an embodiment, the heater is configured to provide thermal energy, for example, in response to a user initiated trigger signal. Microheaters for use in small and stretchable electronics may be found in, e.g., U.S. Patent App. Pub. No. 2013/0140649 and U.S. Patent App. Pub. No. 2013/0041235, each of which is incorporated herein by reference.

For example the internal energy source may be a light source, e.g., an LED or microLED light source, configured to provide stimulus for photoactivation. Examples of light sources suitable for implantation can be found in Kim et al., Science, 340:211-216 (2013); Kim et al., Science 333:838-843 (2011); U.S. Patent App. Pub. No. 2013/0140649 and U.S. Patent App. Pub. No. 2013/0041235, each of which is incorporated herein by reference.

For example, the energy source may be external to the subject and the energy provided transdermally. Energy sources known to be used transdermally include electronic, thermal, light, and magnetic sources. Transdermal energy may be provided by serpentine electronics as in stretchable or epidermal electronics (see, e.g., Kim et al., 2013/0041235, and 2013/0041235, ibid), which may be associated with fabric of clothing or attached to the skin.

In an embodiment, the implantable device includes at least one cartridge or reservoir containing a fluid (e.g., carbon dioxide) that optionally includes a timer or gradual release valve structurally configured to allow release of the fluid over a specific time period. In an embodiment, the release valve is operably connected to control circuitry, which may be programmable. In an embodiment, the cartridge is replaceable. For example, in an embodiment, in response to radiofrequency activation the cartridge is driven to release a gas, e.g., carbon dioxide, or another fluid in pre-determined quantities (e.g., 2 cc amounts). In an embodiment, the cartridge can include a fluid that exists in a first phase while in the fluid cartridge and in a second phase while out of the fluid cartridge. For example the fluid may be liquid while stored in the cartridge and may be released as gas. In an embodiment, the implantable device optionally includes a valve for releasing the fluid. In an embodiment, the implantable device optionally includes a valve for compressing or recondensing the fluid for storage in the cartridge. In an embodiment, the cartridge includes sufficient fluid for multiple successive phase changes for various reversible expansion/contraction events.

In an embodiment, the device includes an outer shell that is structurally permeable or semi-permeable such that the fluid that is added for expansion of the device, for example from the cartridge or reservoir will subsequently permeate through the shell while the device contracts. In an embodiment, the fluid includes a gas. In an embodiment, the gas is generated by phase change from solid or liquid to gas. In an embodiment, the gas may be recondensed when it is desired that the implantable device revert to smaller volume shape or a gas can be released.

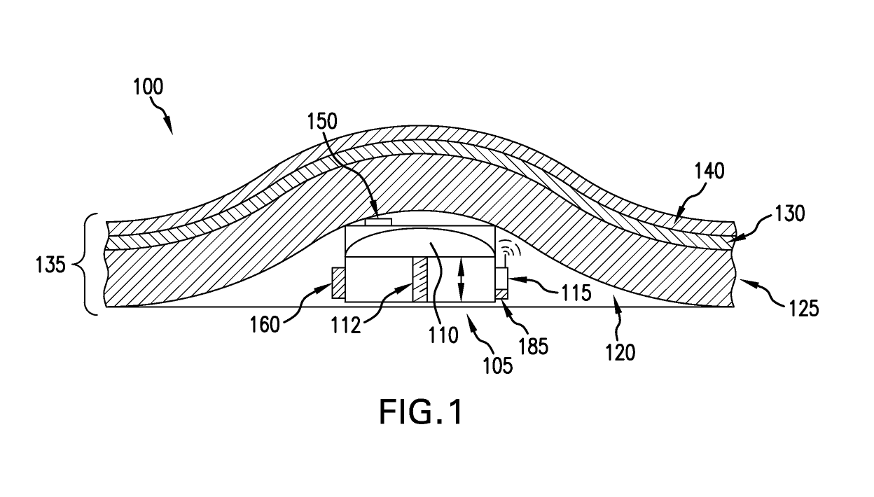

As shown in the Figures, FIG. 1 illustrates a system 100 disclosed herein that includes a “smart” implantable device 105 that is implanted into a subject's body 135 at a space 120 beneath the skin 140, subcutaneous fat 130, and corresponding muscle layer 125. Alternatively, the device is implanted above the muscle layer 125. In an embodiment, the “smart” implantable device 105 includes at least one sensor, a power source (e.g., microbattery) 160, as well as a transceiver and/or receiver and/or optional transmitter component 115, with control circuitry 185 operably coupled to adjustable member 110 that expands and contracts under control circuitry 185, at the direction of a command, a computer program, or one or more optional sensor 150 as a signal received by receiver 115. The “smart” implantable device 105 further includes optional motor 112 to drive adjustable member 110.

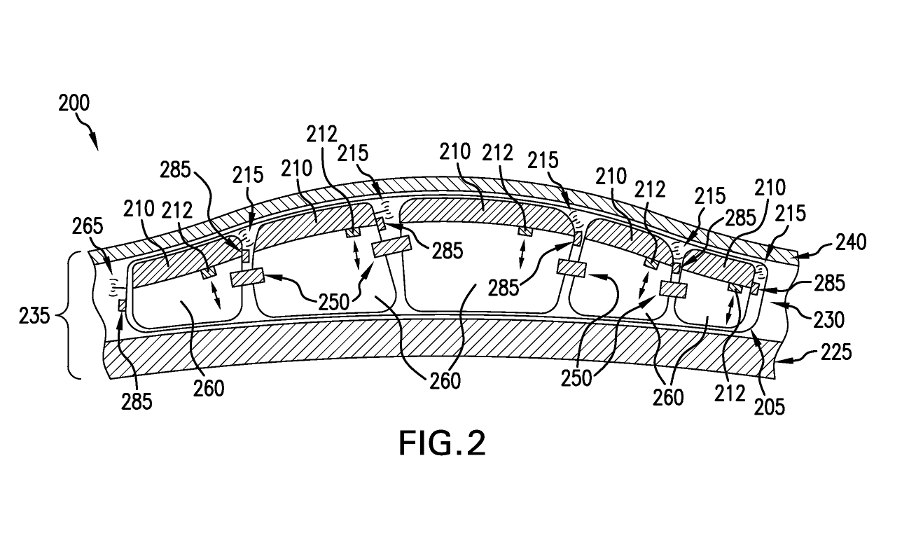

As shown in FIG. 2, a system 200 disclosed herein that includes a “smart” implantable device 205 that is implanted into a subject's body 235 beneath the skin 240 among the subcutaneous fat layer 230, and external to the underlying muscle layer 225. The “smart” implantable device 105 includes control circuitry 285, fluid reservoir 210 regulated by a valve 212, and multiple sections comprising one or more adjustable member 260 that are configured to operate independently or dependently (e.g., by way of a connector 250 such as a valve between one or more sections 260). Each of the multiple sections 260 includes transceiver and/or transmitter and/or receiver component 215 that can be operably coupled to the control circuitry. In an embodiment, the “smart” implantable device outer housing also includes a transceiver and/or receiver and optional transmitter component 265 on the “smart” implantable device coupled to control circuitry. The transmitting/receiving components 215 and 265 are operably coupled to each other, as well as to the adjustable members 210 of each of the multiple sections 260 of the device, which allows for “cross-talk” among sections, as well as with other sources (e.g., sensor, computer program, subject input, etc.). In an embodiment, the fluid adjustable member (reservoir or cartridge) 210 includes a depot or cartridge for storing a fluid, such as liquid or gas, and may be operably coupled to one or more valves 212 or other release ports that cause expansion and contraction of the corresponding section of the device upon receiving a signal (see arrow). Although the fluid reservoir is depicted as being internal to e.g., the adjustable member 260 (which is a section of the whole device that is expandable/contractable), it may instead be adjacent and operably connected to such aspects of the device and may form a distinct component of the device.

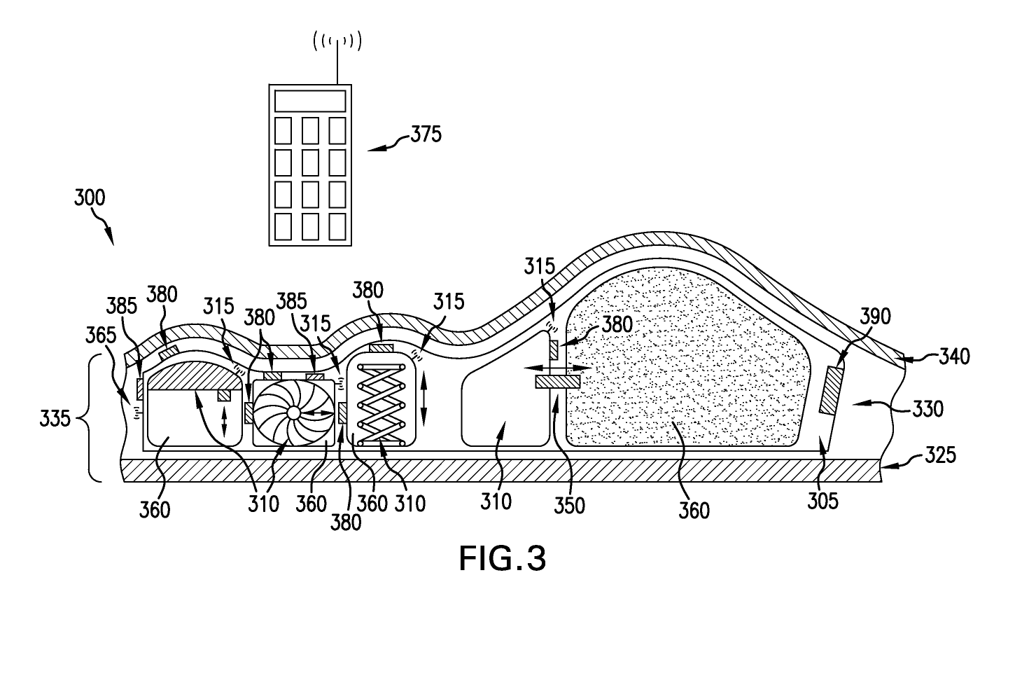

As shown in FIG. 3, a system 300 disclosed herein that includes a “smart” implantable device 305 that is implanted into a subject's body 335 beneath the skin 340, and among the subcutaneous fat layer 330, but external to the underlying muscle layer 325. In an embodiment, the “smart” implantable device 305 includes a means 310 for driving expansion and contraction of adjustable members 360. In an embodiment, in order to produce the desired contour(s), the driving means 310 can include one or more of a liquid reservoir, gas chamber, a motor, an accordion-type expander, or a reservoir that provides a catalyst for responsive element (e.g. a gel) of the adjustable member 360 by way of a connector 350 (e.g., a valve or port). Although the driving means is depicted as being internal to aspects of the device, e.g., the adjustable member 360, it may instead be adjacent and operably connected to such aspects of the device and may form a distinct component of the device. One or more optional sensors 380 present on one or more of the multiple sections of the “smart” implantable device 305, are in operable communication with the transceiver and/or receiver and optional transmitter component 315 of the sections 360 and the adjustable members 310 and are able to detect the expansion and/or contraction of the device (each section or the whole, depending on the location of the sensor). The sensors (depending on location) can also detect physiological (e.g., various biochemical markers or movements, heat, etc.) from the subject's body, that can also be utilized in feedback for controlling the expansion and/or contraction of the device (or sections thereof). Likewise, the transceiver and/or receiver and/or optional transmitter component 365 (e.g., coupled to the device containing the multiple sections) is in operable communication with the transceiver and/or receiver and/or transmitter component 315 of the sections 360 and in operable communication with the adjustable members 310. In an embodiment, one or more of the various mechanical components is operably coupled to control circuitry 385. The “smart” implantable device 305 includes a power source (e.g., battery, capacitor, etc.) 390. The “smart” implantable device 305 can be directed to adjust one or more of the adjustable members 310 by a user controlled remote control 375. A user includes, but is not limited to, the subject itself, a health care worker, computer, or other user.

In an embodiment, the device includes one or more internal linear motors that structurally support the implantable device. In an embodiment, the motors are controllable remotely (e.g., external remote control) and are programmable. In an embodiment, the motors are pre-programmed for a particular profile setting that can be selected by remote control by the subject.

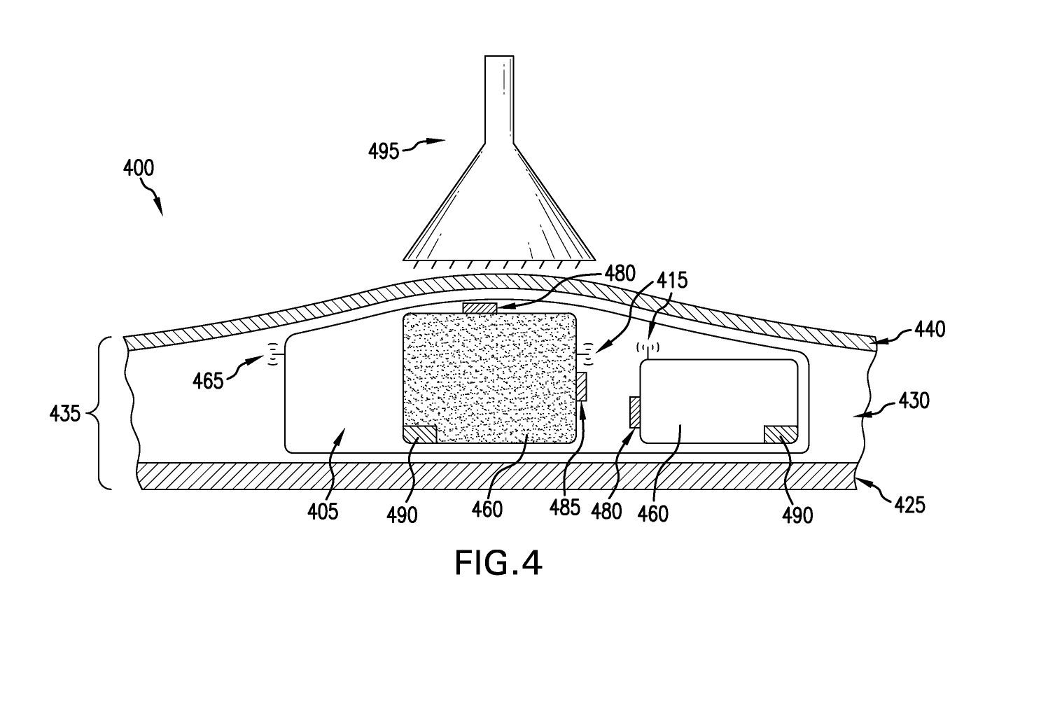

As described in FIG. 4, a system 400 including a “smart” implantable device 405 disclosed herein is implanted into a subject's body 435 beneath the skin 440 among the subcutaneous fat 430 and external to the underlying muscle 425. The “smart” implantable device includes adjustable member 460 comprising a responsive element (e.g., polymer, gas, etc.), transceiver and/or receiver with optional transmitter components 415 for each section or 465 for the outer housing of the device, and control circuitry 485. A power source 490 is contained within one or more of the sections of the “smart” implantable device 405. One or more optional sensors 480 are internal and housed within the “smart” implantable device 405 or external to the device. An energy source 495 is utilized for stimulation of a responsive material to manipulate expansion and/or contraction of the adjustable member 460 of the device 405. Although the energy source 495 is depicted as external to the body, it may be external (transdermal) to the body and device or may be internal to the subject's body and may be integral to the device. For example, a magnetic or ultrasound source 495 configured to provide external or transdermal stimulation for a hydrogel or other filler material is utilized to manipulate expansion and/or contraction of the adjustable member 460 of the device 405.

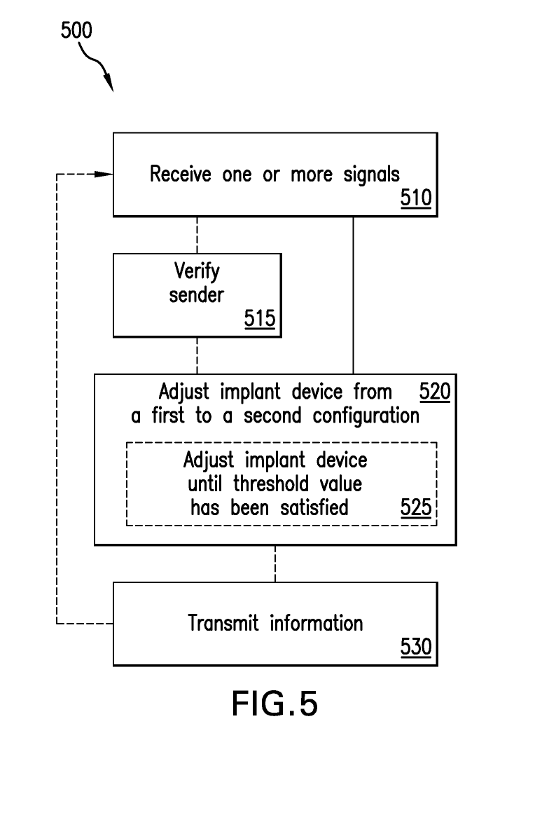

As set forth in FIG. 5, a system 500 disclosed herein, and including a “smart” implantable device operates by receiving one or more signals 510, and responds by adjusting the implantable device from a first configuration to a second configuration 520. Optionally, the implantable device is adjusted until a threshold value has been satisfied 525. Optionally, prior to adjustment, the sender of the one or more signals is verified 515. Next, optionally, information is transmitted 530 to a user (e.g., a person or computer), and the feedback loop is continued as shown (dotted lines back to Receive one or more signals).

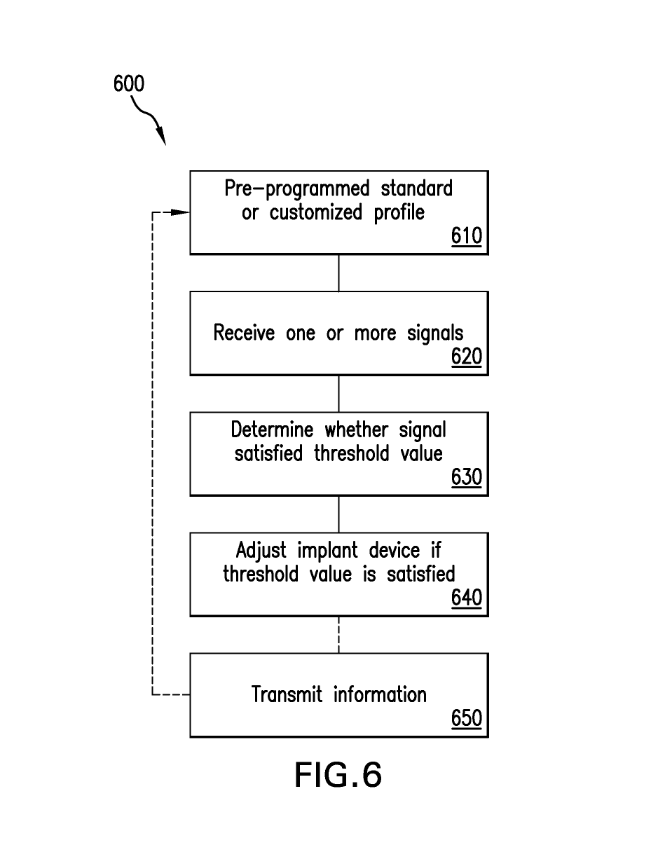

As described in FIG. 6, a system 600 includes a device that operates by including a pre-programmed standard or customized profile 610, receiving one or more signals 620, determining whether the signal satisfies a threshold value 630, and adjusting the implantable device if the threshold value is satisfied 640. Optionally, information is transmitted 650 subsequent to the determination and/or adjustment, and the feedback loop is continued as shown (dotted lines back to Pre-programmed standard or customized profile).

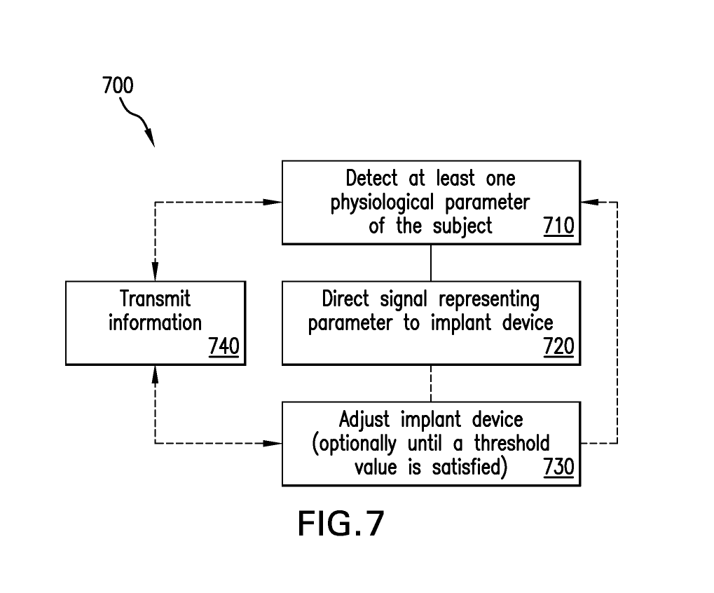

As described in FIG. 7, a system 700 includes a device that operates by detecting at least one physiological parameter of the subject 710. Next, the signal representing the parameter is directed to the implantable device 720. The implantable device is adjusted 730 (optionally until a threshold value is satisfied). Optionally, information is transmitted 740 and the feedback loop continues as shown (see dotted lines).

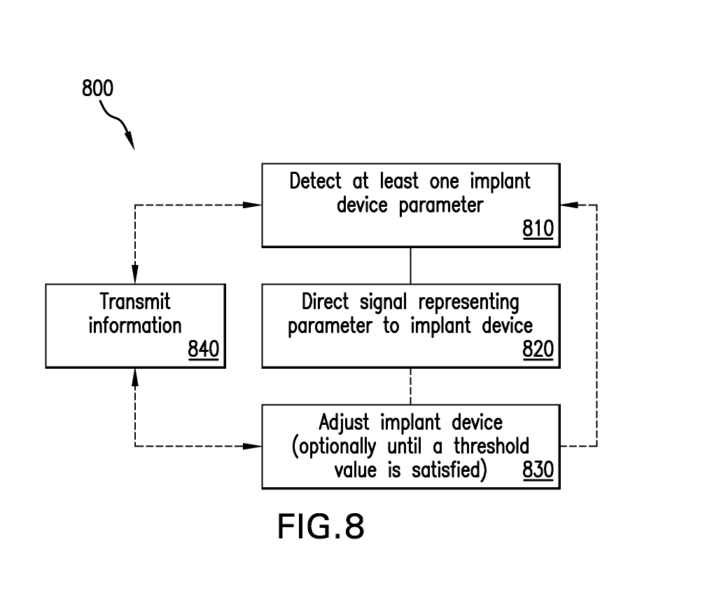

As described in FIG. 8, a system 800 includes a device that operates by detecting at least one implantable device parameter 810. Next, the signal representing the parameter is directed to the implantable device 820. The implantable device is adjusted 830 (optionally until a threshold value is satisfied). Optionally, information is transmitted 840 and the feedback loop continues as shown (see dotted lines).

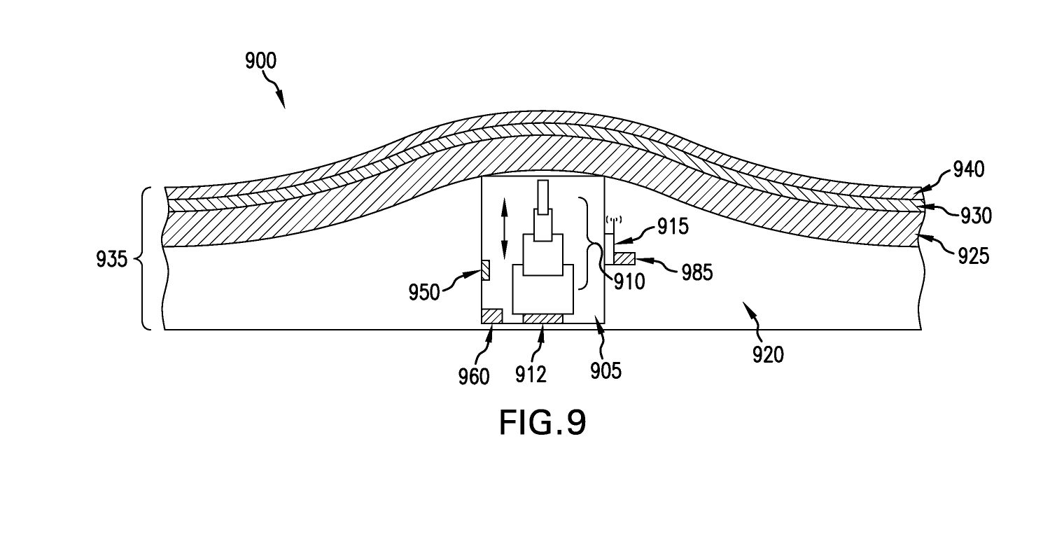

As described in FIG. 9, a system 900 includes a “smart” implantable device 905 for implanting into a subject's body 935 at a space 920 beneath the skin 940, the subcutaneous fat 930, and the muscle 925. The implantable device 905 contains a sensor 950 to detect the expansion or contraction of the device 905. The implantable device 905 includes one or more adjustable members 910 in the form of telescoping members, and a transceiver and/or transmitter and/or receiver component 915, as well as a power source (e.g., battery) 960.

### PROPHETIC EXAMPLES

### Prophetic Example 1

### A Smart Telescoping Tissue Implant System

A computerized tissue implant system is designed to reversibly expand and modify the contour of tissues on the face and other body parts of patients with burns, scars, or other disfigurements. The implant system is also used to make cosmetic enhancements such as fuller lips, reduced wrinkles and the like. The implant system includes a device with a mechanical expansion unit and circuitry to control the expansion unit, including a transceiver, a sensor and a remote control. The implant system is designed to modify the skin and tissue contours in response to signals from internal, external, or environmental sensors. The implant device also communicates wirelessly with external remote controls and mobile computers.

The implant system uses a reversible expansion system which includes a linear motor that drives a telescoping adjustable arm with a shield which presses the tissue above it. See FIG. 1 (adjustable member, 110). A small electric motor drives the adjustable arm upward or downward to expand or contract the tissue contour over the implant device. For example, a piezo-electric motor 2.8 mm×2.8 mm×6 mm with a thrust speed of approximately 10 mm/sec is available from New Scale Technologies, Inc., Victor, N.Y. The motor is powered by a 2.3 volt battery on the device, and both are controlled by circuitry on the device, which receives signals from the system sensors and/or remote controls, and actuates the piezo-electric motor to raise or lower the shield a predetermined distance. The adjustable arm and shield are constructed from a polymer, for example a copolymer of lactide, glycolide and caprolactone (see e.g., Bertleff et al., J. Soc. Lap. Surg., 13:550-554, 2009, which is incorporated herein by reference) using a 3D printer (available from Z Corporation, Burlington, Mass.). The adjustable arm may be approximately 10 mm in length, fully extended, and the shield may be approximately 25 mm in diameter. The adjustable arm is a telescoping arm composed of concentric cylinders of different diameters which collapse or extend upon each other (see FIG. 9). The piezo-electric motor is controlled by circuitry in the device to raise or lower the adjustable arm and the shield (see FIG. 1). The system circuitry is programmed to respond to internal and environmental sensors by raising or lowering the shield a specified distance (e.g., 2 mm) based on sensor signals. For example a distance sensor may detect the distance between the shield and the base of the implanted device. The distance data are transmitted to a transceiver (e.g., micro transceivers are available from Jameco Electronics, Belmont, Calif.) on the device, and control circuitry calculates the height of the shield required to maintain the preferred skin contour. Microsensors to detect displacement, distance, and tilt are available from SignalQuest, LLC, Lebanon, N.H. The implanted device may also contain a timer and programming to adjust the height of the shield as a scar or burn wound heals over days, weeks and months. Alternatively, visual inspection of a scar or burn wound may indicate the implanted device needs to expand or contract to achieve the desired skin and tissue contour. An expansion or contraction signal may be transmitted from a remote control to the implant device transceiver which in turn signals circuitry on the device to activate the piezo-electric motor to raise or lower the telescoping arm and the shield to a prescribed height as specified in the remote control signal.

### Prophetic Example 2

### A Tissue Implant Device with Multiple Reservoirs Containing CO2, which Expand or Contract in Response to Sensor Signals

A tissue implant device is constructed with multiple adjustable sections having reservoirs and valves, CO2 cartridges, and transceivers. The sections have flexible envelopes surrounding the reservoirs to contain CO2 gas, and valves that connect the reservoir to the adjustable sections and valves that vent to the surrounding tissues. The outer envelope is constructed from flexible, biocompatible materials that allow expansion or contraction of each section to achieve any desired contour (see e.g., FIGS. 2 and 3). The implant device has control circuitry including microchips, transceivers, and sensors to respond to external or internal signals, open and close the valves, and contract or expand the sections according to programmed instructions. A 5.0 volt battery empowers the circuitry, the transceivers, and the electronic valves and sensors. The implant device may also be controlled by signals from a remote control operated by the implant user or a second party.

The tissue implant device is constructed with a series of expandable sections having reservoirs that include CO2 cartridges, valves to control the flow of CO2 gas, and transceivers to control each reservoir, as well as a transceiver to control the device. Multiple adjustable sections with flexible walls and ceilings are made from elastic biocompatible polymers using a 3D printing process. 3D printers and methods for 3D printing are available from Stratasys Ltd., Eden Prairie, Minn. For example, a series of linked sections (see FIG. 2) may be printed using polyethylene and expanded polytetrafluoroethylene (EPTFE) to construct the flexible sections (see e.g., U.S. Patent App. Pub. No. 2008/0275569 by Lesh published on Nov. 6, 2008, which is incorporated herein by reference). A framework structure may be printed from a polymer, (e.g., polylactic-co-glycolic acid, PLGA) to provide a framework to support flexible walls and the solenoid valves. CO2 cartridges containing liquefied CO2 are built into each adjustable section. Miniature CO2 cartridges approximately 1 inch in length are available from Leland Ltd, Inc., South Plainfield, N.J. (see e.g., Gas Cartridge Brochure available online at lelandtd.com, the subject matter of which is incorporated herein by reference). Each CO2 cartridge is connected to an electronically controlled solenoid valve to control the flow of CO2 gas into the section. See FIG. 2. For example, an adjustable section of the implant may require approximately 0.10 mL of CO2 gas at approximately 2 lbs. per square inch to inflate the section to a desired size and shape. Each section of the implant device may be expanded or contracted independently since each reservoir includes a transceiver to receive signals from external and internal sensors and/or the transceiver of the device control. For example an external sensor, (e.g., a video camera) may detect an undesirable skin contour on a burn wound over the implanted device. Signals from the video camera are received by transceivers on the implant device, and valves controlling CO2 gas cartridges are opened to inflate the implant reservoirs. The video camera may monitor the skin contours and provide feedback signals to close the valves when sufficient CO2 has been released into the implant reservoirs. Conversely, signaling to electronic valves in the outer wall of the adjustable section(s) may allow CO2 gas to be vented to the tissues surrounding the implant device when contraction of the reservoirs is required. Individual sections may be filled with different volumes and pressures of CO2 gas to create the desired shape. For example, an uneven or undulating implant surface may be required (e.g., see FIG. 3).

The adjustable sections also share a lateral wall, which may contain a connector valve (e.g., miniature solenoid valves are available from Parker Hannifin, Precision Fluidics Div., Hollis, N.H.) that allows CO2 gas transfer between the sections. See FIG. 2. For example, visual inspection of skin contours over the implanted device may suggest equilibrating the CO2 pressure in two adjacent sections to provide the desired shape for the implant and the corresponding skin contour. The user signals the device with a remote control at radiofrequency (RF) wavelengths to open the valve connecting the two adjacent reservoirs and allow flow of CO2 between the adjustable sections. Following visual inspection of the skin contour, the expansion or contraction of the sections may be adjusted further using the remote control to activate valves controlling CO2 gas flow.

The implant device responds to internal sensors to control the shape and size of the implant. For example the implant device may contain pressure sensors to monitor the CO2 pressure in the adjustable sections. Miniature pressure sensors with a pressure range of 0.15 psi to 75.0 psi are available from First Sensor AG, Munich, Germany. Pressure sensors in the adjustable sections may detect and transmit the CO2 pressures in the adjustable sections to control circuitry on the device. For example the implant device may be inflated prior to implantation based on imaging of the implant site (e.g., burn wound, skin transplant, or incision scar), and the CO2 pressure of each adjustable section is stored in memory. Following implantation (device may be either inflated or deflated for implantation), the control circuitry activates the control valves to restore the previously recorded CO2 pressures in each adjustable section. Moreover, the pressure sensors may periodically monitor the CO2 pressures in the adjustable sections, and the control circuitry may adjust them as needed to maintain the desired shape of the implant device.

### Prophetic Example 3

### A Computer-Controlled Cosmetic Implant Device with an Electro-Osmotic Pump

A cosmetic implant device is constructed with adjustable compartments that expand and contract in response to input from a remote control operated by the user. The implant device is constructed with compartments that expand to enhance the appearance of the face, for example by increasing the fullness of the lips. Control circuitry on the device receives signals from a remote control or external or internal sensors, leading to actuation of an electro-osmotic pump on the device, which modulates the shape and size of the adjustable compartment through reversible pumping of electrolyte fluids into the compartment. Expansion and contraction of the adjustable compartment is augmented by the inclusion of a filler material that swells or condenses in response to changes in osmolality (i.e., ionic strength).

The cosmetic tissue implant device is constructed with a series of expandable compartments that include osmotically responsive filler, e.g., a polymer; electronic valves and transceivers, and an electro-osmotic pump. Multiple linked compartments that expand and contract are made from elastic, biocompatible polymers using a 3D printing process. 3D printers and methods for 3D printing are available from Stratasys Ltd., Eden Prairie, Minn. For example, a series of linked adjustable compartments (see FIG. 3) may be printed using polyethylene and expanded polytetrafluoroethylene (EPTFE) to construct the flexible reservoirs (see e.g., U.S. Patent Appl. No. 2008/0275569 by Lesh published on Nov. 6, 2008, which is incorporated herein by reference). A framework structure may be printed from a polymer, (e.g., polylactic-co-glycolic acid, PLGA) to provide shape and to support intra- and extra-compartmental valves (e.g., miniature solenoid valves are available from Parker Hannifin, Precision Fluidics Div., Hollis, N.H.). Each compartment has a valve controlling fluid flow to and from the surrounding tissue, and a valve connecting to a supply reservoir (e.g., see FIG. 3). The supply reservoir (e.g., 310) contains electrolyte to fill the adjustable member compartments 360 when a change in ionic strength is required. Each compartment contains a filler material that responds to changes in osmolality by swelling or contracting. For example, polyacrylate hydrogels that swell and contract in response to physiological salt solutions may be used as fillers in the compartments (see e.g., Horkay et al., Biomacromolecules 1:84-90, 2000 which is incorporated herein by reference). A solution containing monovalent and divalent cations (e.g., Na+, K+ and Ca++, Ba++) may be used to swell or contract the polyacrylate hydrogel fillers and their corresponding adjustable compartments 360. Methods to calculate ionic strength and the corresponding swelling or contraction of hydrogels are described (see Horkay et al., Ibid.). An electro-osmotic pump is incorporated in the implanted device to drive the flow of electrolyte into the compartments of the device. An electro-osmotic pump (EOP) is created by 3D printing of conjugated polymers in the reservoirs of the implant device. For example, electrodes may be printed in the supply reservoir (containing electrolyte) and in the expanding/contracting compartments containing filler. A conjugated polymer blend, for example, poly(3,4-ethylenedioxythiophene (PEDOT) with poly(styrenesulfonate) (PSS) is used to create electrodes that minimize electrolysis and allow—electro-osmotic flow, with approximately a 2 volt potential across the electrodes (see e.g. Erlandsson et al., Electrophoresis 32:784-790, 2011, which is incorporated herein by reference). The electro-osmotic pump, solenoid valves, and control circuitry on the implant device are powered by a microbattery, which is printed using lithium oxide-based inks and 3D printing technology. Methods and materials to make microbatteries approximately 5 mm in length and width with high energy and power densities are described (see e.g., Sun et al., Advanced Materials 25:4539-4543, 2013, which is incorporated herein by reference).

A cosmetic implant device is fabricated to provide shape to the lips, and to allow adjustment of lip fullness following implantation. The implant is printed with multiple compartments, which have different dimensions to conform to the shape of the lips. Photographic images of the individual's lips may be used to print the implant device. For example, the upper lip implant may taper on the ends with compartments that range between 1.5 mm and 5 mm when expanded. For the lower lip, the compartments may be 2 mm to 8 mm in depth and height when fully expanded. 3D printing of the implant device is done with a 3- to 4-fold reduction in the implant dimensions to allow for the expansion of the compartments when electrolyte solution enters and filler material (e.g., polyacrylate hydrogel) swells. The implant device supply reservoirs are filled with approximately 1-5 mL of salt solution, e.g., 5 mM CaCl₂, prior to implantation. Signaling from a remote control activates the EOP to pump 5 mM CaCl₂ into designated compartments to attain a concentration of approximately 1 mM, which causes shrinkage of the filler and reduces the volume of the compartment. Visual inspection of the lips may indicate that more or less fullness is required to improve the appearance of the lips. A remote control is used to signal to the implant device to increase the lower lip size from approximately 3 mm to 5 mm in height in specific compartments. Compartments corresponding to the middle of the implant device are purged of CaCl₂ solution using external solenoid valves (flowing to surrounding tissues), and deionized water is infused into the selected compartments to increase expansion of the polyacrylate hydrogel (see e.g., Horkay et al., Ibid.) and the corresponding compartments. Control circuitry on the implant device includes programs to reduce or increase the volume of individual compartments by 10% to 90% in response to signals from the remote control or sensors in the implant device. Reversible expansion and contraction of the implant device may be automated based on sensors that signal the status of the implant device. For example, pressure sensors in the compartments may indicate the relative inflation or deflation of the compartments and signal the control circuitry to restore preset pressures by electro-osmotic pumping of electrolyte solution, venting of fluid to surrounding tissues, or infusing deionized water into the compartment.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system comprising:
at least one implantable device including a receiver responsive to at least one initiation signal;
circuitry configured for determining a reversible change in configuration of the implantable device in response to the at least one initiation signal, and generating a configuration signal;
at least one fluid-adjustable member operably coupled to the circuitry and responsive to the configuration signal to change the implantable device from a first configuration to a second configuration; wherein
the receiver is responsive to at least one reversion signal; and
circuitry configured for determining a reversion to the first configuration of the implantable device in response to the at least one reversion signal, and generating a reversal signal; wherein
the at least one a fluid-adjustable member operably coupled to the circuitry is responsive to the reversal signal to return the implantable device from a second configuration to the first configuration.

2.-3. (canceled)

4. The system of claim 1, further including at least one sensor operably coupled to the implantable device.

5.-16. (canceled)

17. The system of claim 1, wherein at least one of the initiation signal or the reversion signal is directed by a computer program.

18. The system of claim 17, wherein at least one of the initiation signal or the reversion signal is directed by a timer.

19. The system of 1, wherein at least one of the first configuration or the second configuration includes a change in size or shape of the device.

20. (canceled)

21. The system of claim 1, further including at least one power source internal or external to the implantable device that has been implanted into a subject's body.

22.-30. (canceled)

31. An implantable device, comprising:
a receiver responsive to at least one initiation signal;
circuitry configured for determining a reversible change in configuration of the implantable device in response to the at least one initiation signal to generate a configuration signal;
at least one adjustable fluid cartridge operably coupled to the circuitry and responsive to the configuration signal to change the implantable device from a first configuration to a second configuration; wherein
the receiver is responsive to at least one reversion signal; and
circuitry configured for determining a reversion to the first configuration of the implantable device in response to the at least one reversion signal to generate a reversal signal;
wherein the at least one adjustable fluid cartridge operably coupled to the circuitry is responsive to the reversal signal to return the implantable device from a second configuration to the first configuration.

32.-33. (canceled)

34. The implantable device of claim 31, further including at least one sensor operably coupled to the receiver.

35.-50. (canceled)

51. The implantable device of claim 31, further including at least one energy source internal or external to the implantable device that has been implanted into a subject's body.

52.-61. (canceled)

62. A system comprising:
at least one implantable device including a receiver responsive to at least one initiation signal;
circuitry configured for determining a reversible change in configuration of the implantable device in response to the at least one initiation signal, and generating a configuration signal;
at least one adjustable member including a responsive element contained within the device;
the device operably coupled to the circuitry and responsive to the configuration signal to change the implantable device from a first configuration to a second configuration; wherein
the receiver is responsive to at least one reversion signal; and
circuitry configured for determining a reversion to the first configuration of the implantable device in response to the at least one reversion signal, and generating a reversal signal; wherein
the at least one adjustable member comprising a responsive element operably coupled to the circuitry is responsive to the reversal signal to return the implantable device from a second configuration to the first configuration.

63.-64. (canceled)

65. The system of claim 62, further including at least one sensor operably coupled to the at least one adjustable member.

66.-74. (canceled)

75. The system of claim 62, wherein at least one of the initiation signal or the reversion signal is directed by a computer program.

76. The system of claim 75, wherein at least one of the initiation signal or the reversion signal is directed by a timer.

77.-85. (canceled)

86. The system of claim 62, wherein the device is configured to change color upon pH change.

87. The system of claim 62, wherein at least one of the initiation signal or the reversion signal is directed by at least one sensor.

88.-89. (canceled)

90. The system of claim 87, wherein the sensor includes at least one of a heart rate detector, body temperature detector, blood pressure detector, blood sugar sensor, pupillometer, or other physiological sensor.

91.-93. (canceled)

94. An implantable device, comprising:
a receiver responsive to at least one initiation signal;
circuitry configured for determining a reversible change in configuration of the implantable device in response to the at least one initiation signal to generate a configuration signal;
at least one responsive gel or polymer contained within the device;
the device operably coupled to the circuitry and responsive to the configuration signal to change the implantable device from a first configuration to a second configuration; wherein
the receiver is responsive to at least one reversion signal; and
circuitry configured for determining a reversion to the first configuration of the implantable device in response to the at least one reversion signal to generate a reversal signal; wherein
the at least one adjustable reservoir operably coupled to the circuitry is responsive to the reversal signal to return the implantable device from a second configuration to the first configuration.

95.-96. (canceled)

97. The implantable device of claim 94, further including at least one sensor operably coupled to the receiver.

98.-101. (canceled)

102. The implantable device of claim 94, wherein at least one of the initiation signal or the reversion signal is generated by an external control.

103.-105. (canceled)

106. The implantable device of claim 94, wherein the device includes at least one electroactive polymer configured for expansion and contraction of the volume of the device.

107.-115. (canceled)

116. The implantable device of claim 94, wherein the implantable device includes a plurality of separate sections.

117. The implantable device of claim 116, wherein at least one of the plurality of separate sections are independently controllable.

118.-130. (canceled)
